(19) United States
(12) Patent Application Publication
(10) Pub. No.: US 2025/0011464 A1
(43) Pub. Date: Jan. 9, 2025

(54) HIGH-EFFICACY LABELING OF ANTIBODIES WITH OLIGONUCLEOTIDE LABELS

(71) Applicant: ACTOME GMBH, Freiburg (DE)

(72) Inventor: Csaba JENEY, Freiburg (DE)

(21) Appl. No.: 18/710,047

(22) PCT Filed: Nov. 14, 2022
(86) PCT No.: PCT/EP2022/081746

§ 371 Date: May 14, 2024

(30) Foreign Application Priority Data
Nov. 15, 2021 (GB) 2116442.1

(51) Int. Cl. C07K 16/32; C07K 1/13

(52) U.S. Cl. CPC C07K 16/32; C07K 1/13; C07K 2317/24

(57) ABSTRACT

The invention comprises a method for highly efficient selective labelling of antibodies with oligonucleotides. The method may involve the selective removal of glycans from the antibody and replacing them by artificial glycosides, especially N-glycosides including oligonucleotides. The process is believed to be facilitated by the oligonucleotide induced orientation of the antibody C′E loop and to a lesser extent by the Cγ2-Cγ3 domain. The method results in the high efficacy labelling of antibodies.

The invention comprises a method for highly efficient selective labelling of antibodies with oligonucleotides. The method can include the selective removal of glycans from the antibody and replacing them by artificial glycosides, especially N-glycosides including oligonucleotides.

The process is believed to be facilitated by the oligonucleotide induced orientation of the antibody C′E loop and to a lesser extent by the Cγ2-Cγ3 domain. The method results in the high efficacy labelling of antibodies.

The two major approaches of antibody-DNA conjugation are non-covalent (for example biotin-streptavidin) or covalent conjugation methods. Non-covalent methods are prone to label dissociation during applications resulting in label hopping and short shelf-life. Covalent conjugation methods are differentiated according to site-specific and non-selective conjugation. An additional aim is the modification of antibodies to obtain labeled antibodies or antibodies with a strictly defined number of DNA-labels. The degree of labeling influences many applications of antibody-DNA conjugations, especially labeling strategies involving unique molecular identifiers. The random labeling strategies are hampered by the statistical behaviour of the conjugation reactions, a degree of one label per antibodies results in conjugates containing a mixture of mono-labeled antibodies, double-labeled antibodies, and unlabeled ones. This behaviour is shared by both the non-covalent (biotin-streptavidin) or covalent conjugation methods, as both approaches have a statistical reaction step. Strategies using site-specific conjugation are capable of producing antibody-DNA conjugates with a defined number of DNA-labels. However, if the conjugation reaction is not exhaustive (an efficiency of 100%), this introduces also the undesired statistical behavior.

Short oligonucleotide sequences (below 30 bp), show no facilitated conjugation reaction and still exert statistical conjugation (McMahon NP, Jones JA, Kwon S, et al. Oligonucleotide conjugated antibodies permit highly multiplexed immunofluorescence for future use in clinical histopathology. J Biomed Opt. 2020;25(05):1. doi:10.1117/1.jbo.25.5.056004).

Consequently, a method achieving exhaustive DNA conjugation of antibodies is highly desirable.

Chemoselective ligation reactions used in the selective targeting of small molecules suffer from a significant limitation—the intrinsic kinetic properties of bimolecular covalent reactions. The current chemoselective ligation reactions are characterized by a bimolecular rate constant of the order of 10⁴-10⁵ M⁻¹ min⁻¹ (Jencks, W. P. (1959) J. Am. Chem. Soc. 81, 475-481, Sayer, J. M., Peskin, M. and Jencks, W. P. (1973) J. Am. Chem. Soc. 95, 4277-4287). As the covalent reactions have relatively low rate constants, high concentrations of the reagents are required for the reactions to proceed at a useful rate. The chemoselective conjugation of biological molecules is increasingly less effective with the increasing molecular weights of the reactants due to the achievable low concentration of the reactants, diffusion limits, steric problems and unfavorable interactions between the reaction partners. Maerle et al. have demonstrated the antibody conjugation reaction with an oligonucleotide of 60 bp has unacceptable low yield. (Maerle A V., Simonova MA, Pivovarov VD, et al. Development of the covalent antibody-DNA conjugates technology for detection of IgE and IgM antibodies by immuno-PCR. PLoS One. 2019; 14(1):1-19. doi:10.1371/journal.pone.0209860) The efficacy of the high-yield conjugation reactions, including simple click-chemistry reactions, used to conjugate oligonucleotides to antibodies using non-site directed approaches is varied and typically less than 30%, and as low as only 5% for oligonucleotides larger than 50 bp. (Wiener J, Kokotek D, Rosowski S, Lickert H, Meier M. Preparation of single- and double-oligonucleotide antibody conjugates and their application for protein analytics. Sci Rep. 2020; 10(1):1-12. doi:10.1038/s41598-020-58238-6).

Approaches of selective carbohydrate modification are diverse from chemical, enzymatic oxidation of sugars (Wilchek and Bayer 1987; Bayer et al. 1988), and enzymatic transfer of a fluorescence-marked sugar by a glycosyltransferase (Gross and Brossmer 1988; Gross and Brossmer 1991). Strategies for the metabolic incorporation of sialic acid derivatives into glycoconjugates of the cell surface have also been developed (Kayser et al. 1992; Keppler et al. 1995; Lee et al. 1999; Lemieux and Bertozzi 1998; Schmidt et al. 1998). The chemical synthesis of different activated sugars and their enzymatic transfer starting from L-galactose was described by Hallgren and Hindsgaul (1995). Methods where a biotinylated sugar was transferred to an N-acetyllactosamine derivative with a fucosyl transferase, additionally UDP-GaINAc and its transfer using azide as an biorthogonal label were also developed (Hang et al. 2003). Finally a strategy was created to transfer the nucleotide sugar of UDP-Gal with a keto modification using a mutant of the recombinant bovine β4Gal-T1 (Khi dekel et al. 2003).

The low bimolecular rate constants of the chemoselective ligation reactions can be overcome if they are preceded by a fast, non-covalent association of the reactive partners. An example of this principle, when an aptamer is functionalized with a reactive electrophilic group and bound through noncovalent association to the target protein (elastase) on neutrophil cell surfaces is described by Charlton, J., Sennello, J. and Smith, D. (1997) Chem. Biol. 4, 809-816). Once bound, a reactive phosphonate ester forms an irreversible covalent adduct with a serine residue within the enzyme's active site. However, it needs a complex and unique design of labels for each target and no such strategy was devised for antibodies and labels having structural elements other than small reactive chemoselective chemical groups.

Simulations show high amplitude motions of Fc N-glycan of IgG antibodies, where glycan residues are dynamically binding and detaching from the Fc surface, exposing the glycan termini to the bulk solvent for enzymatic modification in the open conformation. This is consistent with NMR studies on Fc glycan accessibility having two distinct states: one with the glycan has interactions with the polypeptide surface and buried and the other when the glycan is freely exposed to solvent and exempt from the glycan-polypeptide interactions. (Lee HS, Im W. Effects of N-Glycan Composition on Structure and Dynamics of IgG1 Fc and Their Implications for Antibody Engineering. Sci Rep. 2017; 7(1):1-10. doi:10.1038/s41598-017-12830-5). This behaviour has been detected with different glycan forms and can be extended to other sugar containing compounds with a similar molecular weight, such as the natural glycans including N-glycosides.

The invention provides the first method for the selective, exhaustive labeling of antibodies with oligonucleotides. It is advantageous in applications where exhaustive labeling of oligonucleotide-conjugated antibodies is necessary such as methods that are used to translate the detection signal from the protein to the DNA level, high multiplexing strategies for protein analytical assays (Ali, M. M. et al. Rolling circle amplification: a versatile tool for chemical biology, materials science and medicine. Chem. Soc. Rev. 43, 3324-3341 (2014), Karakus U, Thamamongood T, Ciminski K, et al. MHC class II proteins mediate cross-species entry of bat influenza viruses. Nature. 2019. doi:10.1038/s41586-019-0955-3; Lundberg, M., Eriksson, A., Tran, B., Assarsson, E. & Fredriksson, S. Homogeneous antibody-based proximity extension assays provide sensitive and specific detection of low-abundant proteins in human blood. Nucleic Acids Res. 39, e102-e102 (2011)). They are useful when combined with next generation sequencing (NGS) technologies or sequential fluorescence hybridization methods, so proteins can be quantitated in parallel with e.g. CITEseq or SABER. (Stoeckius, M. et al. Simultaneous epitope and transcriptome measurement in single cells. Nat. Methods 14, 865-868 (2017); Saka, S. K. et al. Immuno-SABER enables highly multiplexed and amplified protein imaging in tissues. Nat. Biotechnol. 37, 1080-1090 (2019)). All of these applications need chemically defined antibody conjugates, with known labeling valency and efficacy, to produce consistent numerical results.

The invention is also advantageous in any method using a binding scheme to facilitate effective conjugation by applying a high rate non-covalent binding step between an antibody C′E loop or/and Cγ2-Cγ3 domain and the payload such as an oligonucleotide.

The object of the invention is to provide a method of selective, highly efficient labelling of antibodies with oligonucleotides. This can be achieved by using a concentration of at least 1 μM oligonucleotide in the reaction mixture which allows affinity binding of the antibody with the oligonucleotide to occur. This can also be achieved for example by installation of a conjugation-capable (handle) sugar moiety to asparagine-297 of IgG or conformationally related amino acids of IgG, including endoglycosidase based removal of the native conserved asparagine N-glycan after the innermost GIcNAc moiety if present, ensuring the facilitated binding of functionalized N-glycosides and reacting the functionalized N-glycoside payload with the conjugation handle in order to overcome the limitation of statistical conjugation reactions.

The payload is a high molecular weight moiety where the efficient conjugation reaction is limited by the physical, chemical or conformational effects.

The payload is advantageously an oligonucleotide or a modified oligonucleotide. The oligonucleotide may be attached to a further labelling moiety such as a fluorophore

In a first aspect, the invention provides a process for labelling an antibody with an oligonucleotide, said process comprising
(a) Reacting the antibody with an oligonucleotide conjugated to a reactive group, to form a covalent linkage between the oligonucleotide and the antibody;
wherein the concentration of the oligonucleotide conjugate is at least 1 μM.

At a physiological pH, antibodies have an affinity for oligonucleotides. This affinity binding is sufficient to hold the oligonucleotide in place, which increases the efficiency of the subsequent conjugation reaction between the reactive group linked to the oligonucleotide and a group on the antibody, covalently linking the oligonucleotide to the antibody.

As used herein, the term “antibody” is used interchangeably with “immunoglobulin” and encompasses polyclonal antibodies, monoclonal antibodies, multispecific antibodies such as bispecific antibodies, chimeric antibodies, humanized antibodies, human antibodies, and any other modified immunoglobulin molecule comprising an antigen recognition site so long as the antibodies exhibit the desired biological activity, and contain at least the CH2 portion of at least one heavy chain. Preferably the antibody is a monoclonal antibody. An antibody can be a member of any of the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Preferably, the antibody is an IgG antibody, more preferably an IgG1 or IgG4. The term “antibody” is also intended to include conjugates of the antibody, for example conjugates with polyethylene glycol, PEG.

Further, except where the context requires otherwise, the term “antibody” should be understood to encompass complete antibodies and antibody fragments comprising an antigen-binding region of the complete antibody and the CH2 domain, including scFv-CH2-CH3 fusion proteins. An antibody can be produced by a hybridoma, or by synthetic means such as recombinant DNA techniques, phage display or yeast display technologies or using transgenic mice, or liquid or solid phase peptide synthesis.

The antibody is preferably an IgG antibody. Preferably the antibody is commercially available. Preferably the antibody is known to be used in a diagnostic method. Preferably the antibody is known to be used in therapy. Suitable antibodies for use in the invention include trastuzumab, pertuzumab and combinations thereof.

The concentration of the oligonucleotide in the reaction mixture is at least 1 μM. Preferably the concentration of the oligonucleotide is at least 10 μM, or at least 50 μM, or at least 70 μM, or at least 100 μM, or at least 500 μM, more preferably concentration of the oligonucleotide is at least 1 mM. This concentration of the oligonucleotide increases the speed of covalent bonding resulting in nearly 100% antibodies being labelled. “Nearly 100%” or “as close to 100%” of the antibodies being labelled as used herein means over 90%, preferably over 95%, more preferably over 98%, most preferably over 99% of the antibodies are labelled. It is thought that the oligonucleotide diffuses into the pocket between the heavy chains, and forms non-covalent links. This increases the concentration of the oligonucleotide present within the pocket, which improves the kinetics of the covalent bonding reaction. The low bimolecular rate constants of the chemoselective ligation reaction are overcome as it is preceded by a fast, non-covalent association of oligonucleotide.

Preferably each heavy chain within the antibody is labelled with an oligonucleotide. Thus where the antibody molecule has two heavy chains, it is labelled with 2 oligonucleotides. It is desirable to have as close to 100% antibodies labelled as possible. It can be difficult to separate the unlabeled antibody from the labeled antibody. The presence of the unlabeled antibody reduces the signal generated and increases the background noise.

Oligonucleotides used to label the antibody are generally at least 30 bases in length, for example at least 40 bases in length, at least 50 bases in length, at least 60 bases in length at least 70 bases in length, at least 80 bases in length, at least 90 bases in length or at least 100 bases in length. Preferably, the oligonucleotide contains 40-150 nucleotides, for example 50-120, or 60-100 nucleotides.

The oligonucleotides may comprise RNA or DNA, and be single or double stranded. The nucleotides that form the nucleic acid can be chemically modified for example, to increase the stability of the molecule, to improve its bioavailability, to confer additional activity on it and/or provide additional negative charge. For example, the pyrimidine bases may be modified at the 6 or 8 positions, and purine bases at the 5 position with CH₃ or halogens such as I, Br or Cl.

Modifications of pyrimidines bases also include N²H₃, O⁶ —CH₃, N⁶ —CH₃ and N² —CH₃. Modifications at the 2′ position of the sugar moiety, “sugar modifications”, typically add a NH₂, F or OCH₃ group. Modifications can also include 3′ and 5′ modifications such as capping.

Alternatively modified nucleotides, such as morpholino nucleotides, locked nucleic acids (LNA) and peptide nucleic acids (PNA) can be used. Morpholino oligonucleotides are assembled from different morpholino subunits, each of which contains one of the four genetic bases (adenine, cytosine, guanine, and thymine) linked to a 6-membered morpholine ring. The subunits are joined by non-ionic phosphorodiamidate intersubunit linkages to give a morpholino oligonucleotide. LNA monomers are characterised in that the furanose ring conformation is restricted by a methylene linker that connects the 2′-0 position to the 4′-C position. PNA is an analogue of DNA in which the backbone is a pseudopeptide rather than a sugar.

The reactive group may be attached directly to the oligonucleotide. Alternatively, the reactive group may be attached to the oligonucleotide via a spacer. The spacer may be selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups. Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S and NR₁₁, wherein R₁₁ is independently selected from the group consisting of hydrogen, halogen, hydroxy, C₁-C₂₄ alkyl groups, C₆-C₂₄ (hetero)aryl groups, O₇-C₂₄ alkyl(hetero)aryl groups and C₇-C₂₄ (hetero)arylalkyl groups. Most preferably, the heteroatom is O.

Suitable spacers include (poly)ethylene glycol diamines (e.g. 1,8-diamino-3,6-dioxaoctane or equivalents comprising longer ethylene glycol chains), polyethylene glycol or polyethylene oxide chains, polypropylene glycol or polypropylene oxide chains and 1,x-diaminoalkanes wherein x is the number of carbon atoms in the alkane. Preferably the spacer is a polyethylene glycol, more preferably 2,2′-[Oxybis(2,1-ethandiyloxy)]diethanol (PEG4).

Preferably, the invention provides a process for labelling an antibody with an oligonucleotide comprising
(a) deglycosylating a conserved N-glycosylated amino acid within a CH2 domain of antibody heavy chain;
(b) Reacting the deglycosylated antibody with an oligonucleotide conjugated to a reactive group to form a covalent linkage between the oligonucleotide and the deglycosylated antibody;
wherein the concentration of the oligonucleotide conjugate is at least 1 μM.

The term “glycan” herein refers to a monosaccharide or oligosaccharide chain that is linked to a protein. The glycan is attached to a protein via the C-1 carbon of one sugar, which may be without further substitution (monosaccharide) or may be further substituted at one or more of its hydroxyl groups (oligosaccharide). Typically, a naturally occurring glycan comprises 1 to about 10 saccharide moieties.

A glycan of a glycoprotein may be a monosaccharide. Typically, a monosaccharide glycan of a glycoprotein consists of a single N-acetylglucosamine, glucose, mannose or fucose covalently attached to the protein.

A glycan may also be a linear or branched oligosaccharide. The sugar that is directly attached to the protein is called the core sugar. A sugar that is not directly attached to the protein and is attached to at least two other sugars is called an internal sugar. A terminal sugar is a sugar that is not directly attached to the protein but is bound to a single other sugar, i.e. carrying no further sugar substituents at one or more of its other hydroxyl groups.

For the avoidance of doubt, there may exist multiple terminal sugars in an oligosaccharide of a glycoprotein, but only one core sugar. The end of the oligosaccharide that is directly attached to the protein is called the reducing end. The other end of the oligosaccharide is called the non-reducing end of the glycan.

A glycan may be an O-linked glycan, an N-linked glycan or a C-linked glycan. In an N-linked glycan, a monosaccharide or oligosaccharide glycan is bonded to the protein via an N-atom in an amino acid of the protein, typically via an amide nitrogen in the side chain of asparagine (Asn) or arginine (Arg). For example, the glycan is attached to the protein via an an N-glycosidic bond. The asparagine that is substituted with the glycan on its side-chain is typically part of the sequence Asn-X-Ser/Thr, with X being any amino acid but proline and Ser/Thr being either serine or threonine. A wide diversity of chains exist for N-linked glycans.

N-glycosylation of immunoglobulins is well known. Immunoglobulins are generally made up of two heavy chains and two light chains linked by disulfide bonds. The chains are subdivided in constant and variable domains. There are a number of known conserved N-glycosylation sites on asparagines within the constant regions, in particular the constant regions of the heavy chains. Where the antibody or immunoglobulin comprises two heavy chains, then preferably both heavy chains are glycosylated i.e. at least one glycan is present on each heavy chain. The location of the glycosylation site within the CH2 region varies depending on the isotype. For example, in IgG immunoglobulins, the conserved N glycosylation site is Asparagine 297 (amino acid number according to common literature), for IgA1 it is Asparagine 144, for IgA2 Asparagine 133 and/or Asparagine 205; for it is IgM Asparagine 207; for IgE Asparagine 146 and Asparagine 252 can be glycosylated; and for IgD the glycosylation site is Asparagine 225. Preferably if the immunoglobulin comprises two heavy chains, then both heavy chains are glycosylated at the one or more conserved N glycosylation sites.

The N-glycosylated amino acid is preferably Asparagine 297.

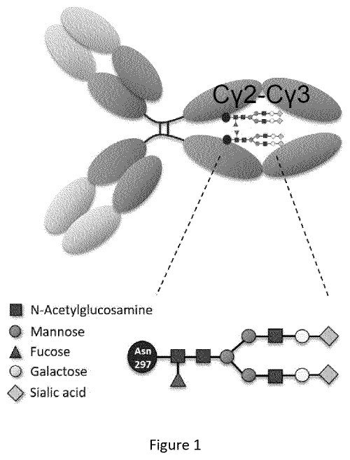

As shown in FIG. 1, the N-glycosylated amino acid is attached to a glycan with an innermost core N-acetylglucosamine (GIcNAc) moiety attached directly to the amino acid. This GIcNAc moiety is joined to a number of galactose, mannose, fructose and/or further N-acetylglucosamine moieties to form the glycan. As an initial step the antibody can be deglycosylated to remove one or more of the saccharide units from the glycan, preferably leaving the innermost or core N-acetylglucosamine. Preferably only the innermost core N-acetylglucosamine remains.

The deglycosylation can be carried out using an enzyme such as a sialidase and/or galactosidase and/or endoglycosidase. An endoglycosidase can be used to remove the native conserved asparagine N-glycan after the innermost GIcNAc moiety. Suitable endoglycosidases include Peptide-N-Glycosidae F (PNGase F), endoglycosidase F (Endo F) including Endo F1, Endo F2, and Endo F3, endoglycosidase H (Endo H), endoglycosidase S (Endo S) and endoglycosidase S2 (Endo S2). Particularly preferred are the Fc specific endoglycosidase EndoS2 (Genovis) or EndoS (New England Biolabs) which hydrolyze the Fc glycans to the innermost N-acetylglucosamine (GIcNAc) moiety on several subclasses and species of IgG.

Alternatively, antibodies which only have a single N-acetylglucosamine moiety attached to the N-glycosylated amino acid can be formed by expression of a monoclonal antibody in the presence of swainsonine or in an engineered host organism.

It is thought that the removal of the glycan provides an improved binding surface within the antibody for nucleic acids. The absence of the glycan allows increased affinity binding between the antibody and the oligonucleotide. This affinity binding effectively holds the oligonucleotide in place and so enhances the efficiency of the subsequent reaction which covalently links the oligonucleotide to the modified deglycosylated antibody.

In one option the reactive group on the oligonucleotide is crosslinked to the antibody. Methods of carrying out suitable crosslinking reactions and suitable reactive groups are known in the art, as set out in ThermoScientific Crosslinking technical handbook (2012) “Easy molecular bonding crosslinking technology Reactivity chemistries, applications and structure references.” The oligonucleotide may be conjugated to a reactive group capable of reacting with a primary amine or sulfhydryl group within the antibody to form a covalent bond linking the oligonucleotide to the antibody. Antibodies (in particular IgG antibodies) generally have 10 to 15 readily available lysine amines that can react with the reactive group to bind the oligonucleotide. Alternatively, a photochemical reaction can be carried out to link the oligonucleotide to the antibody through a nucleophilic or active hydrogen group.

Suitable reactive groups capable of reacting with a primary amine include isothiocyanates, Isocyanates, acyl azides, NHS esters, sulfonyl chlorides, aldehydes, glyoxals, epoxides, oxiranes, carbonates, aryl halides, imido esters, carbodiimides, anhydrides and fluorophenyl esters. Preferably the reactive group is an NHS ester or imidoester.

Suitable reactive groups capable of reacting with a sulfhydryl group include haloacetyls, maleimides, aziridines, acrylic alloys, arylation agents, vinyl sulfones, pyridyl disulfides, TNB thiols, and disulfide reducing agents.

Suitable photoreactive groups include arylazides, azidomethylcoumarins, benzophenones, anthraquinones, certain diazo compounds, diazirines, and psoralen derivatives.

Amine-reactive reactive groups Primary amines exist at the N-terminus of each polypeptide chain and in the side chain of lysine (Lys, K) amino acid residues. These primary amines are positively charged at physiological pH, so they occur predominantly on the outer surfaces of native protein tertiary structures, where they are easily accessible to conjugation reagents that are introduced into the aqueous medium. Primary amines are particularly nucleophilic, making them easy for conjugation with several reactive groups, including isothiocyanates, Isocyanates, acyl azides, NHS esters, sulfonyl chlorides, aldehydes, glyoxals, epoxides, oxiranes, carbonates, aryl halides, imido esters, carbodiimides, anhydrides and fluorophenyl esters. Most of these conjugate to amines either by acylation or alkylation. NHS esters and imidoesters are the most popular amine-specific functional groups that are incorporated into protein crosslinking and labeling reagents.

### NHS Ester

NHS esters are reactive groups that are formed by carbodiimide activation of carboxylate molecules. NHS ester-activated crosslinkers react with primary amines under physiological to slightly alkaline conditions (pH 7.2 to 9) to form stable amide bonds. The reaction releases N-hydroxysuccinimide (NHS). NHS ester crosslinking reactions are most commonly carried out in phosphate, carbonate-bicarbonate, HEPES or borate buffers at pH 7.2 to 8.5 for 0.5 to 4 hours at room temperature (20° C.) or 4° C.

Sulfo-NHS esters are identical to NHS esters except that they contain a sulfonate group (—SO₃) on the N-hydroxysuccinimide ring. This charged group has no effect on reaction chemistry, but does tend to increase the water solubility of crosslinkers containing it. Additionally, the charged group prevents sulfo-NHS crosslinkers from penetrating cell membranes, so they can be used in cell surface crosslinking procedures. Examples of NHS and sulfo-NHS are Disuccinimidyl suberate (DSS) and bis(sulfosuccinimidyl)suberate (BS3 or sulfo-DSS) amine-to-amine crosslinkers. DSS is not directly soluble in water, but once it is dissolved it can penetrate through the cell membranes in order to act within the cells. BS3 is water soluble, but when charged it cannot penetrate cell membranes which limit BS3 crosslinking to the surface of intact cells.

### Imidoester

Imidoester crosslinkers react with primary amines to form amidine bonds. Imidoester crosslinkers react quickly with amines at alkaline pH, but have short half-lives. As the pH becomes more alkaline, the half-life and reactivity with amines increase; therefore crosslinking is more effective when carried out at pH 10 than at pH 8. Reaction conditions below pH 10 can lead to side reactions, although amidine formation is favored between pH 8-10. Studies with monofunctional alkyl imidates show that a conjugation with only one functional imidoester group can form at pH<10. An intermediate N-alkyl imidate forms in the lower pH range and is either crosslinked with another amine in the immediate vicinity, which leads to N, N′-amidine derivatives, or it is converted into an amidine bond.

### Sulfhydryl Reactive Crosslinking

Sulfhydryls, also called thiols, are found in proteins in the side chain of cysteine (Cys, C) amino acids. Pairs of cysteine sulfhydryl groups are often linked by disulfide bridges (—S—S—) within or between polypeptide chains as the basis of the native tertiary or quaternary protein structure. Typically, only free or reduced sulfhydryl groups (—SH) are available for the reaction with thiol-reactive compounds.

Sulfhydryls are present in most proteins, but not as abundant as primary amines, so crosslinking via sulfhydryl groups is more selective and precise. Sulfhydryl groups in proteins are often involved in disulfide bonds, so crosslinking at these sites typically does not significantly modify the underlying protein structure or block binding sites. The number of available (i.e., free) sulfhydryl groups can be easily controlled or modified; they can be generated by reducing native disulfide bridges or introduced into molecules by reaction with primary amines using sulfhydryl addition reagents such as 2-iminothiolane (Traut's reagent), N-succinimidyl S-acetylthioacetate (SATA), N-Succinimidyl S-Acetylthiopropionate (SATP) or N-Succinimidyl S-acetyl(thiotetraethylene glycol) (SAT (PEG) 4).

Sulfhydryl reactive chemical groups are well known in the art and include haloacetyls, maleimides, aziridines, acrylic alloys, arylation agents, vinyl sulfones, pyridyl disulfides, TNB thiols, and disulfide reducing agents. Most of these groups conjugate to sulfhydryls by either alkylation (usually forming a thioether bond) or disulfide exchange (forming a disulfide bond).

Haloacetyls have the formula R—NH(C═O)CH₂Halo where R is the oligonucleotide and optional linker.Preferred haloacetyls include an iodoacetyl or a bromoacetyl group.

Malemide groups have the structure H₂C₂(CO)₂NR where R is the oligonucleotide and optional linker. The maleimide group reacts specifically with sulfhydryl groups when the pH of the reaction mixture is between 6.5 and 7.5 and results in the formation of a stable thioether linkage.

Pyridyl disulfides have the structure R—S—S—(C₅H₅N) where R is the oligonucleotide and optional linker. Pyridyl disulfides react with sulfhydryl groups over a broad pH range (the optimum is pH 4 to 5) to form disulfide bonds. During the reaction, a disulfide exchange occurs between the molecule's —SH group and the reagent's 2-pyridyldithiol group. As a result, pyridine-2-thione is released and can be measured spectrophotometrically (Aₘₐₓ=343 nm) to monitor the progress of the reaction.

### Photoreactive Crosslinking

Photoactivatable (or photochemical) crosslinking reactions require energy from light to initiate the reaction. Photoreactive groups are chemically inert compounds that become reactive when exposed to ultraviolet or visible light. Virtually all types of photoreactive groups used in reagents for crosslinking applications must be exposed to ultraviolet light (UV light) to activate the molecule. The use of photoreactive groups can be advantageous as the oligonucleotide can be added to the antibody and left to allow affinity binding with the antibody to occur. The mixture can then be exposed to UV light to cause the formation of the covalent link between the antibody and the oligonucleotide.

Photoreactive groups that can be used as the reactive group conjugated to the oligonucleotides to form a covalent linkage to the antibody include arylazides, azidomethylcoumarins, benzophenones, anthraquinones, certain diazo compounds, diazirines, and psoralen derivatives. The preferred groups are arylazides and diazirines.

Arylazides comprise an aryl group substituted with an azide group(N═N═N), The arylazide is conjugated to the oligonucleotide. Upon exposure to UV light a nitrene group is formed which can initiate addition reactions with double bonds or insertion into CH and NH sites. Diazirine groups have the formula R—CHN₂, where R is the oligonucleotide and optional linker. When exposed to UV light a carbene intermediate forms, which can form a covalent bond with an amino acid side chain containing a nucleophilic or active hydrogen group.

Alternatively, if the antibody has been deglycosylated in the process of the present invention, part or all of the glycan can be replaced with an oligonucleotide. Methods for carrying out this process are known in the art, including the method described in U.S. Pat. No. 99,873,736. This method has been previously used to attach small molecules to the glycan. It has not been used previously to attach large molecules such as oligonucleotides.

In another aspect, the invention relates to a method comprising (a) installation of a conjugation-capable sugar, including endoglycosidase based removal of the native conserved asparagine N-glycan after the innermost GIcNAc moiety if present, (b) binding of functionalized N-glycosides and (c) reacting the functionalized N-glycosides payload with the conjugation handle.

Thus, the method may comprise the steps of
(a) deglycosylating a conserved N-glycosylated amino acid within a CH2 domain of antibody heavy chain;
(b) attaching a functional group to the deglycosylated antibody to form a modified antibody;
(c) Reacting the modified antibody with an oligonucleotide conjugated to a reactive group to form a covalent linkage between the oligonucleotide and the modified antibody;
wherein the reactive group reacts with the functional group to form a covalent linkage; and
wherein the concentration of the oligonucleotide conjugate is at least 1 μM.

The antibody is modified by attaching a functional group. Preferably, a modified GIcNAc moiety is formed by attaching a sugar moiety comprising the functional group. This can be achieved by contacting the antibody with a sugar moiety with one or more functional groups in the presence of an enzyme which attaches the sugar moiety, preferably to an N-acetylglucosamine moiety on the antibody. Suitable enzyme include galactosyltransferases, for example p(1,4)-galactotransferases, p(1,3)-N-galactotransferases, β(1,4)-galactotransferases. The β(1,4)-galactotransferases may contain a mutant catalytic domain such as those described in U.S. Pat. No. 7,482,133 including GaIT(Y289L), GaIT(Y2891), and GaIT(Y289N).

As used herein, the term “sugar” is used to refer to a monosaccharide, and the term “sugar moiety” refers to a monosaccharide derivative, which contains one or more functional groups. The sugar moiety is preferably derived from a sugar or an amino sugar such as galactose, mannose, glucose, glucosamine, galactosamine, N-acetylglucosamine, N-acetylgalactosamine, fucose or sialic acid. More preferably, the sugar moiety is preferably derived from galactose, galactosamine, mannose, glucose, N-acetylgalactosamine or N-acetylglucosamine.

The sugar moiety may additionally comprise a nucleotide, such as a nucleoside monophosphate, or a nucleoside diphosphate. Examples of such nucleotides include uridine diphosphate (UDP), guanosine diphosphate (GDP), thymidine diphosphate (TDP), cytidine diphosphate (CDP) and cytidine monophosphate (CMP). Preferably, the nucleotide is UDP. Suitable nucleoside monophosphates and nucleoside diphosphates are described in WO 2009/102820.

Examples of sugar moieties comprising a nucleotide include Uridine 5′-diphospho-N-acetylazidogalactosamine (GaINAz-UDP), UDP-N-acetyl-alpha-D-galactosamine (6-AzGal-UDP), 6-AzGaINAc-UDP, Uridine 5′-diphospho-N-acetylazidogalactosamine (4-AzGaINAz-UDP), UDP-6-azido-6-deoxy-D-glucose (6-AzGalNAz-UDP), UDP-6-azido-6-deoxy-D-glucose (6-AzGlc-UDP), 6-AzGlcNAz-UDP, 2-ketoGal-UDP, 2-N-propionylGalNAc-UDP,2-(but-3-yonic acid amido)-2-deoxy-galactose-UDP, 6-chloro-6-deoxygalactose-UDP (6-CIGal-UDP), 6-thio-6-deoxygalactose-UDP (6-HSGal-UDP) 2-chloro-2-deoxygalactose-UDP (2-CIGal-UDP), 2-thio-2-deoxygalactose-UDP (2-HSGal-UDP), 6-chloroacetamido-6-deoxygalactose-UDP (6-GalNAcCI-UDP), 6-thioacetamido-6-deoxygalactose-UDP (6-GalNAcSH-UDP), 2-chloroacetamido-2-deoxygalactose-UDP (2-GalNAcCI-UDP), 2-thioacetamido-2-deoxygalactose-UDP (2-GalNAcSH-UDP), 3-thiopropanoylamido-2-deoxygalactose-UDP (2-GalNProSH-UDP) and 4-thiobutanoylamido-2-deoxygalactose-UDP (2-GaINBuSH-UDP). Preferably the sugar moiety is Uridine 5′-diphospho-N-acetylazidogalactosamine (GaINAz-UDP) or 6-AzGaINAc-UDP.

Preferably, the sugar moiety is a galactosamine derived moiety which is enzymatically attached using a β-1,4-Galactosyltransferase.

The enzymatic attachment of the functional group is typically carried out at 4 to 50° C., preferably between 1° and 45° C., most preferably between 3° and 37° C.

The enzymatic attachment of the functional group is typically carried out at about pH4 to about pH9, preferably between pH5.5 and pH 8.0, most preferably between pH 6 to pH7.5.

As used herein, the “functional group” may be selected from an azido group, a keto group, an alkynyl group, a thiol group or a precursor thereof, a halogen, a sulfonyloxy group, a halogenated acetamido group, a mercaptoacetamido group and a sulfonylated hydroxyacetamido group.

As used herein, an “azido group” is defined as —N₃Rₙ,wherein each R is independently selected from the group consisting of hydrogen, halogen and an (optionally substituted) C₁-C₆ alkyl group, and n is 0,1 or 2. Preferably R is H and n is 1 or 2. More preferably n is 0.

Suitable azide containing sugar moieties include Uridine 5′-diphospho-N-acetylazidogalactosamine (UPD-GaINAz).

As used herein, an “keto group” is defined as a —R₁(O)R₂ group, wherein R₁ is an optionally substituted C₁-C₆ alkyl group, and R₂ is independently selected from the group consisting of hydrogen, halogen, and optionally substituted C₁-C₆ alkyl group. Preferably, R₂ is hydrogen.

As used herein, an “thiol group” is defined as a —R₂SH group, wherein R₂ is an optionally substituted C₁-C₆ alkyl group. Preferably R₃ is C₁, C₂, C₃ or C₄ alkyl group.

As used here in, a “precursor of a thiol group” is herein defined as —R₃SC(O)CH₃ group, wherein R₃, as well its preferred embodiments, are as defined above for a thiol group. Most preferably, said thiol-precursor is —CH₂CH₂CH₂SC(O)CH₃, —CH₂CH₂ SC(O)CH₃, —CH₂SC(O)CH₃ or —SC(O)CH₃, preferably —SC(O)CH₃. During the process for the preparation of a modified glycoprotein, the thiol-precursor is converted to a thiol group.

As used herein, an “sulfonyloxy group” is defined as a —R₄OS(O)₂R₅ group, wherein R₄ is an optionally substituted C₁-C₆ alkyl group, and R₅ is independently selected from the group consisting of C₁-C₆ alkyl group, C₆-C₂₄ aryl groups, O₇—C₂₄ alkylaryl groups and C₇—C₂₄ arylalkyl groups. Preferably R₄ is C₁, C₂, C₃ or C₄ alkyl group. R₆ is preferably a C₁-C₄ alkyl group, a C₆-C₁₂ aryl group, a C₇-C₁₂ alkylaryl group or a C₇-C₁₂ arylalkyl group. More preferably R₅ is C₁, C₂, C₃ or C₄ alkyl group, a phenyl group or a p-tolyl group. Most preferably the sulfonyloxy group is a mesylate group, —OS(O)₂ CH₃, a benzenesulfonate group (—OS(O)₂ (C₆H₅) or a tosylate group (—OS(O)₂ (C₆H₄CH₃).

As used herein, an “halogenated acetamido group” is defined as an —NHC(O)R₆X group, wherein R₆ is an optionally substituted C₁-C₆ alkyl group, and X is F, Cl, Br or I. Preferably R₆ is hydrogen or a C₁, C₂, C₃ or C₄ alkyl group. Preferably, X is Cl or Br, more preferably X is Cl.

As used herein, “mercaptoacetamido” is defined as an —NHC(O) R₇ SH group, wherein R₇ is an optionally substituted C₁-C₆ alkyl group. Preferably R₇ is a C₁, C₂, C₃ or C₄ alkyl group.

Preferred examples include a mercaptoethanoylamido group, a mercaptopropanoylamido group, a mercaptobutanoylamido group and a mercapto-pentanoylamido group, preferably a mercaptopropanoylamido group.

As used herein, an “sulfonylated hydroxyacetamido” is defined as a —NHC(O) R₉ OS(O)₂ R₁₀ group, wherein R₉ is an optionally substituted C₁-C₆ alkyl group and R₁₀ is independently selected from the group consisting of C₁-C₆ alkyl group, C₆-C₂₄ aryl groups, O₇—C₂₄ alkylaryl groups and C₇-C₂₄ arylalkyl groups. Preferably R₉ is C₁, C₂, C₃ or C₄ alkyl group. R₁₀ is preferably a C₁-C₄ alkyl group, a C₆-C₁₂ aryl group, a C₇-C₁₂ alkylaryl group or a C₇-C₁₂ arylalkyl group. More preferably R₁₀ is C₁, C₂, C₃ or C₄ alkyl group, a phenyl group or a p-tolyl group. Most preferably the sulfonyloxy group is a mesylate group —OS(O)₂CH₃, a benzenesulfonate group —OS(O)₂(C₆H₅) or a tosylate group —OS(O)₂(C₆H₄CH₃).

As used herein, an “halogen” is defined as fluorine, chlorine, bromine, or Iodine. Preferably the halogen is chlorine or bromine.

As used herein as “aryl” group refers to a C₆ to C₁₂ monocyclic or bicyclic structure. Optionally, the aryl group may be substituted by one or more substituents. Examples of aryl groups are phenyl and naphthyl.

As used herein as “arylalkyl” and “alkylaryl” refer to groups that comprise at least seven carbon atoms and may include monocyclic and bicyclic structures. Optionally, the arylalkyl groups and alkylaryl may be substituted by one or more substituents. An arylalkyl group is for example benzyl. An alkylaryl group is for example 4-t-butylphenyl.

As used herein, an “alkyl” group may contain 1-8 carbon atoms. It may be linear or branched or cyclic. Unsubstituted alkyl groups may also contain a cyclic moiety. Optionally, the alkyl groups are substituted by one or more substituents. Preferably the alkyl is a C₁, C₂, C₃ or C₄ alkyl group. Examples of alkyl groups include methyl, ethyl, propyl, 2-propyl, t-butyl, pentyl, cyclopentyl, 1-hexyl, cyclohexyl etc.

As used herein, an “alkynyl” group comprises a carbon-carbon triple bond and may contain 2-10 carbon atoms. A terminal alkynyl is an alkynyl group wherein the triple bond is located at a terminal position of a carbon chain. Optionally, the alkynyl group is substituted by one or more substituents, and/or interrupted by heteroatoms selected from the group of oxygen, nitrogen and sulphur. Examples of alkynyl groups include ethynyl, propynyl, butynyl, octynyl, etc.

As used herein, an “cycloalkynyl” group is a cyclic alkynyl group and may contain 3-10 carbon atoms. Optionally, a cycloalkynyl group is substituted by one or more substituents. An example of a cycloalkynyl group is cyclooctynyl. A preferred cycloalkynyl group is dibenzocyclooctyne.

As used herein, a “heterocycloalkynyl” group is a cycloalkynyl group interrupted by heteroatoms selected from the group of oxygen, nitrogen and sulphur. Optionally, a heterocycloalkynyl group is substituted by one or more substituents. An example of a heterocycloalkynyl group is azacyclooctynyl.

As used herein “optionally substituted” means that the group may be substituted with one or more substituents selected from the group consisting of hydroxy, C₁-C₁₂ alkyl groups, C₂-C₁₂ alkenyl groups, C₂-C₁₂ alkynyl groups, C₃-C₁₂ cycloalkyl groups, C₅-C₁₂ cycloalkenyl groups, C₈-C₁₂ cycloalkynyl groups, C₁-C₁₂ alkoxy groups, C₂-C₁₂ alkenyloxy groups, C₂-C₁₂ alkynyloxy groups, C₃-C₁₂ cycloalkyloxy groups, halogens, amino groups, oxo and silyl groups. As used herein the silyl groups can be represented by the formula (R₁₂)₃ Si—, wherein R₁₂ is independently selected from the group consisting of C₁-C₁₂ alkyl groups, C₂-C₁₂ alkenyl groups, C₂-C₁₂ alkynyl groups, C₃-C₁₂ cycloalkyl groups, C₁-C₁₂ alkoxy groups, C₂-C₁₂ alkenyloxy groups, C₂-C₁₂ alkynyloxy groups and C₃-C₁₂ cycloalkyloxy groups, wherein the alkyl groups, alkenyl groups, alkynyl groups, cycloalkyl groups, alkoxy groups, alkenyloxy groups, alkynyloxy groups and cycloalkyloxy groups are optionally substituted, and wherein the alkyl groups, the alkoxy groups, the cycloalkyl groups and the cycloalkoxy groups are optionally interrupted by one of more hetero-atoms selected from the group consisting of O, N and S.

The modified antibody which is defined herein as comprising the functional group is reacted with an oligonucleotide conjugated to a reactive group to form a covalent linkage between the oligonucleotide and the modified antibody. The oligonucleotide becomes conjugated to the modified antibody via a linker comprising the covalent bond formed by the reaction of the functional group and the reactive group.

The reaction to join the oligonucleotide and the modified antibody is typically carried out at 4 to 50° C., preferably between 1° and 45° C., most preferably between 3° and 37° C. Typically the reaction is carried out overnight at 4° C.

The reaction to join the oligonucleotide and the modified antibody is typically carried out at about pH4 to about pH9, preferably between pH5.5 and pH 8.0, most preferably between pH 6 to pH7.5.

The reactive group reacts with the functional group to form a covalent bond. Suitable reactive groups which form a covalent bond with the functional group on the modified antibody are known in the art, for example those described in U.S. Pat. No. 9,987,373.

When the functional group is an azido group, linking of the modified antibody and the oligonucleotide conjugate preferably takes place via a cycloaddition reaction. The reactive group is then preferably selected from the group consisting of alkynyl groups, preferably terminal alkynyl groups, and cycloalkynyl groups. Similar reactions can take place if the reactive group and functional groups are reversed so that the reactive group is an azido group and the functional group is selected from the group consisting of alkynyl groups, preferably terminal alkynyl groups, and cycloalkynyl groups. Preferred cycloalkynyl groups include dibenzocyclooctyne.

When the functional group is a keto group, linking of the modified antibody with the oligonucleotide conjugate preferably takes place via selective conjugation with hydroxylamine derivatives or hydrazines, resulting in respectively oximes or hydrazones. The reactive group is then preferably a primary amino group, e.g. an —NH₂ group, an aminooxy group, e.g. —O—NH₂, or a hydrazinyl group, e.g. —N(H)NH₂. Similar reactions can take place if the reactive group and functional groups are reversed so that the reactive group is keto group and the functional group is a primary amino group.

When the functional group is an alkynyl group, linking of the modified antibody with the oligonucleotide conjugate preferably takes place via a cycloaddition reaction, preferably a 1,3-dipolar cycloaddition. The reactive group is then preferably a 1,3-dipole, such as an azide, a nitrone or a nitrile oxide. Similar reactions can take place if the reactive group and functional groups are reversed so that the reactive group is an alkynyl group and the functional group is a 1,3-dipole.

When the functional group is a thiol group, linking of the modified antibody with the oligonucleotide conjugate preferably takes place via a Michael-type addition reaction. The reactive group is then preferably an N-maleimidyl group for the Michael-type addition, a halogenated acetamido group for the nucleophilic substitution reaction, or a terminal alkene for the thiol-ene reaction. Similar reactions can take place if the reactive group and functional groups are reversed so that the reactive group is a thiol group and the functional group is a N-maleimidyl group, a halogenated acetamido group, or a terminal alkene.

When the functional group is halogen, a halogenated acetamido group, a sulfonyloxy group or a sulfonylated hydroxy acetamido group, linking of the modified antibody with the oligonucleotide conjugate preferably takes place via reaction with a thiol to form a thioether. The reactive group then preferably comprises a thiol group. Similar reactions can take place if the reactive group and functional groups are reversed so that the reactive group is halogen, a halogenated acetamido group, a sulfonyloxy group or a sulfonylated hydroxy acetamido group and the functional group is a thiol.

However, reactive group may also comprise an alcohol group or an amine group.

In one preferred option, the functional group and reactive group are selected from an azido group and a cycloalkynyl group or alkynyl group; a thiol group and an N— malemide group, iodoacetamide, chloroacteamide or disulfide group; a ketone or aldehyde group and a hydrazine, acylhydrazine, aniline or alkoxyamine.

Preferably, the functional group and reactive group are selected from an azido group and a cycloalkynyl group, preferably wherein the cycloalkynyl group is a cyclooctyne group, more preferably dibenzocyclooctyne.

In another aspect the invention provides an antibody labelled with an oligonucleotide obtained by a process according to the invention.

In another aspect the invention provides a kit for use in a process of the invention for labelling an antibody with an oligonucleotide comprising:
(i) An oligonucleotide conjugated to a reactive group; and optionally
(ii) A compound comprising a functional group capable of forming a modified GlcNAc moiety, wherein the functional group and reactive group are capable of reacting to form a covalent bond; and/or
(iii) A Fc specific endoglycosidase; and/or
(iv) a β-1,4-Galactosyltransferase.

Preferred embodiments of the kit are as described for the process of the invention. The kit may further comprise instructions for carrying out the method of the invention as described herein.

The method according to the present invention is capable of achieving 90% or greater efficacy of antibody conjugation for oligonucleotides larger than 50 bp. Preferably the method achieves an efficacy of 95% or greater, 97.5% or greater, 99% or greater, most preferably 100%, 100% efficacy means that each antibody present is labelled with at least one oligonucleotide. Preferably each heavy chain within an antibody molecule is labelled with at least one oligonucleotide

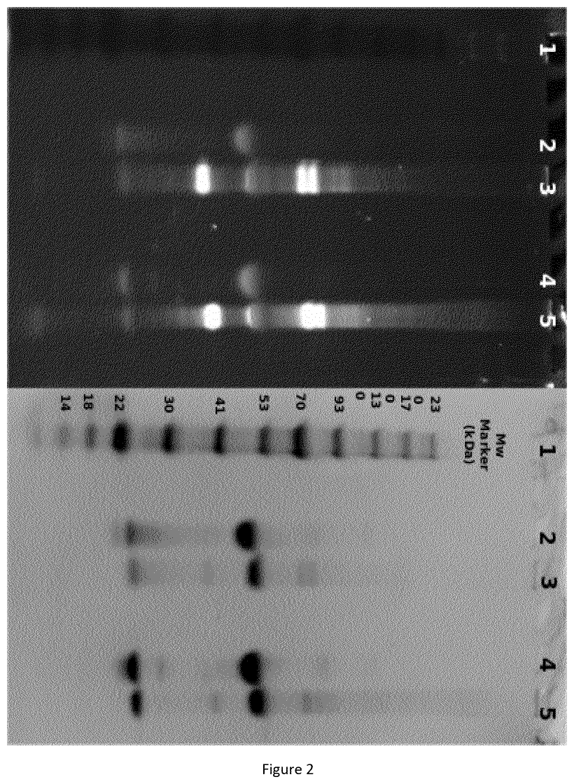

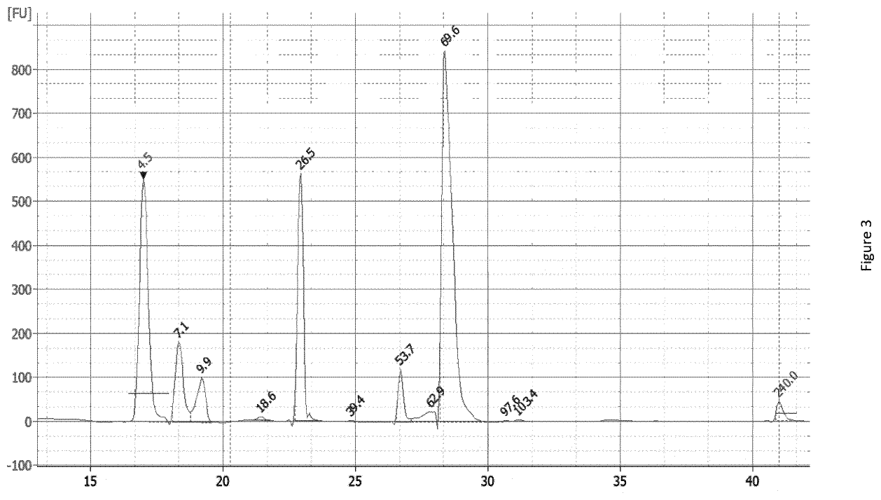

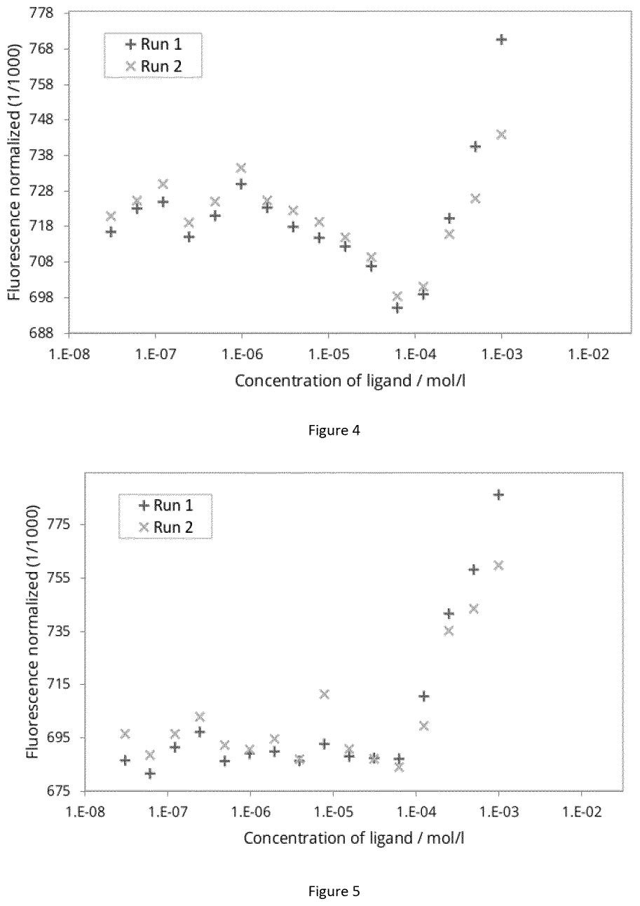

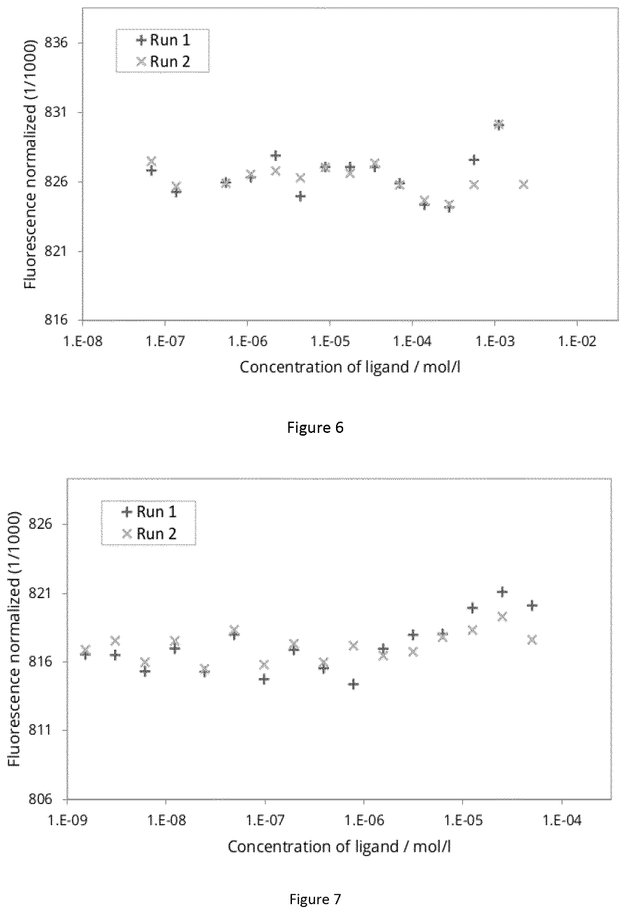

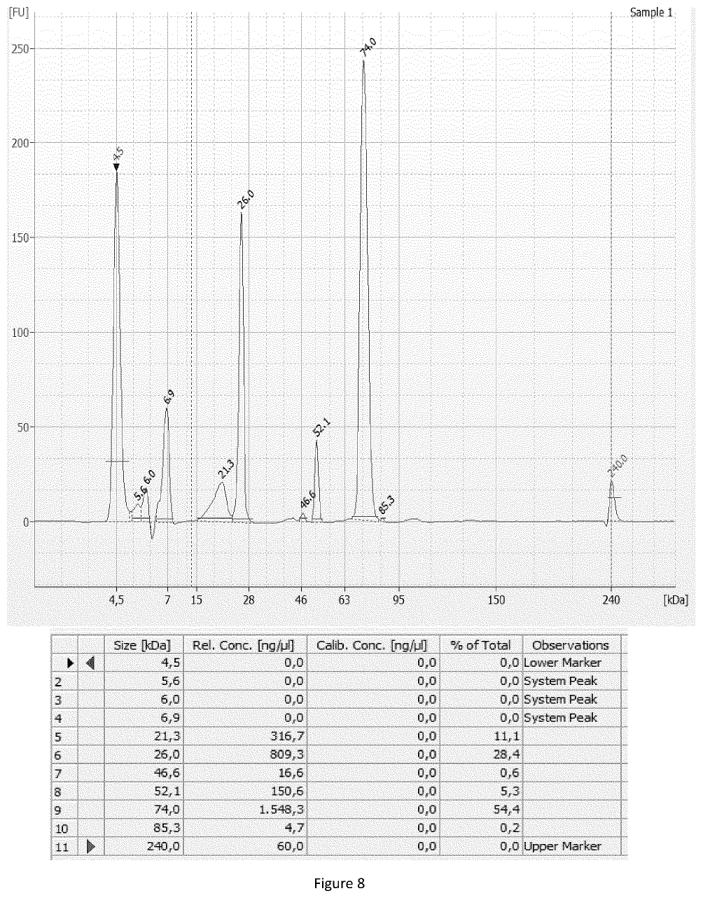

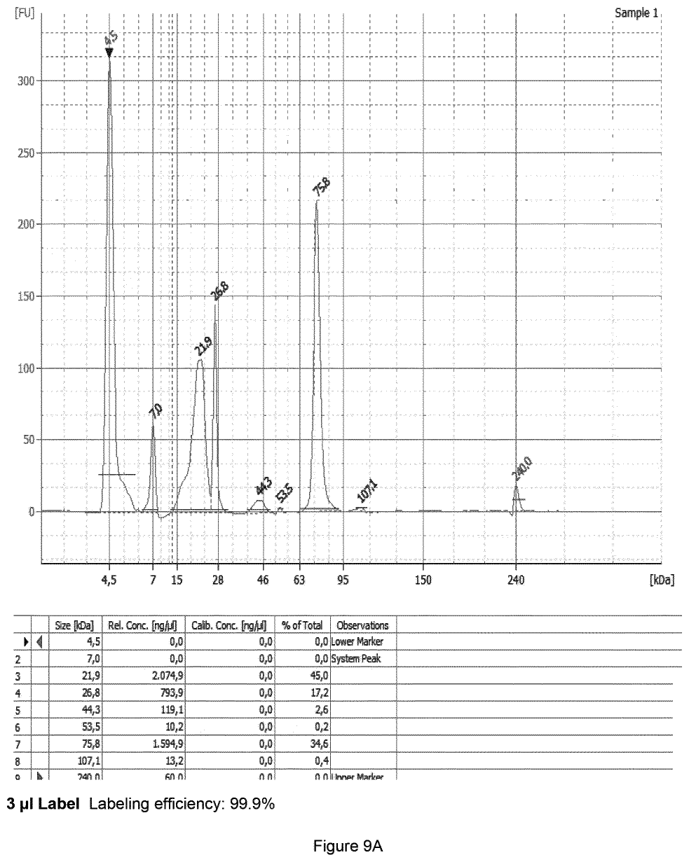

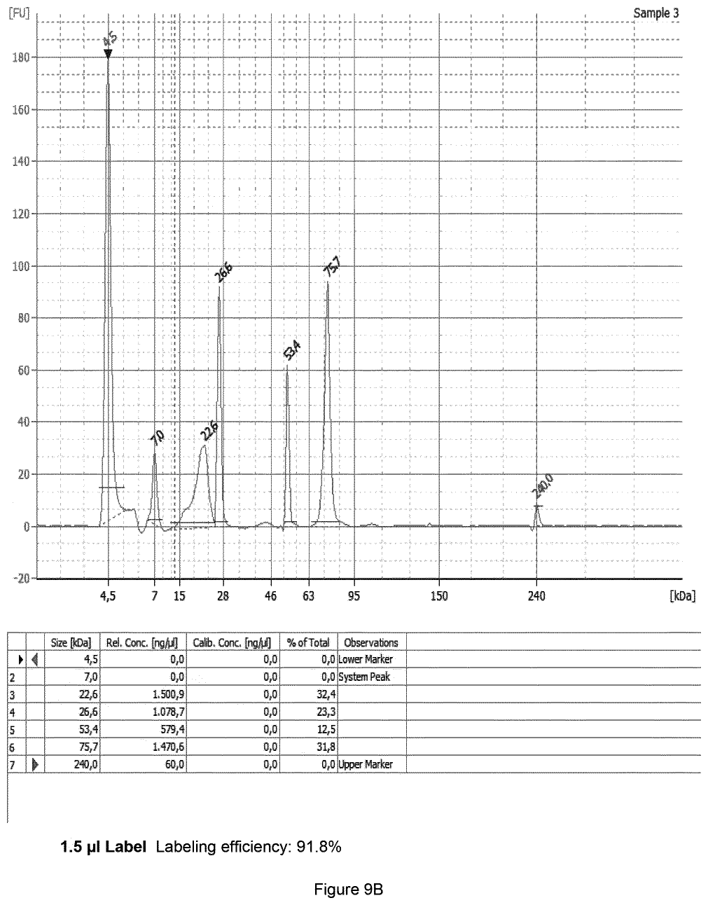

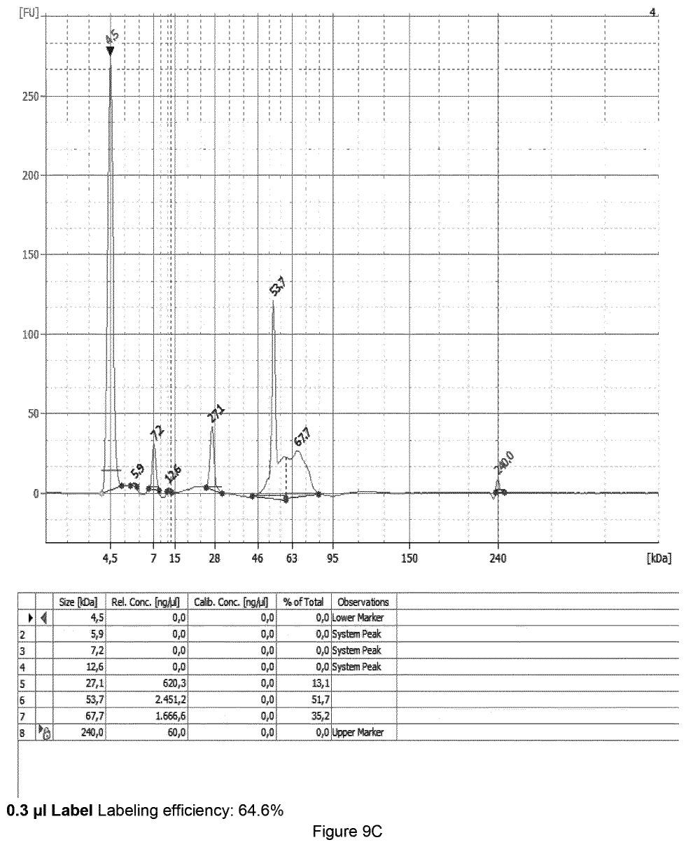

The invention will now be exemplified with reference to the following figures:
FIG. 1 shows the N-glycosylation of antibodies on the asparagine 297 residue. The Cγ2-Cγ3 domain has contacts with the glycosides
FIG. 2 shows the covalent non-selective conjugation of trastuzumab and pertuzumab.
FIG. 3 shows Highly efficient DNA conjugation of trastuzumab antibody.
FIG. 4 shows MST measurement according to Table 1 conditions
FIG. 5 shows MST measurement according to Table 2 conditions
FIG. 6 shows MST measurement according to Table 3 conditions
FIG. 7 shows MST measurement according to Table 4 conditions
FIG. 8 shows oligonucleotide labeled trastuzumab according to the protocol of the example resulting in 99.2% labeling efficiency. The peak at 26 kD indicates the light chain, peak at 52.1 kD indicates unlabeled heavy chain of the antibody and the labeled heavy chain of the antibody is at 74 kD.
FIG. 9 shows the labelling efficiency as different concentrations of the oligonucleotide label.

### EXAMPLES

Example 1 DNA labelling of trastuzumab antibody

The rebuffering of antibodies is usually necessary before carbohydrate processing. 150 μg of trastuzumab antibody in solution is added to a 100 kDa centrifugal ultrafiltration unit (Amicon) mounted on a collection tube. After adding the antibody, the filter unit was filled to 500 μl with TBS buffer (200 mM Tris; 1500 mM NaCl; pH 7.6) and centrifuged at 14,000 g for 3 min at 4° C. The flow-through was discarded. 500 μl of TBS was added, and the filter was centrifuged once more at 14,000 g for 10 min at 4° C., and the flow-through was discarded. Turning the filter upside down in a fresh collection tube and it was centrifuged briefly (1000 g, 20 sec) to collect the rebuffered antibody sample. The sample should have a minimum volume of about 30 μl. If not, add enough TBS to obtain a final volume of 30 μl.

The modification of the carbohydrates on antibody hinge region starts with the deglycosylation of the asparagine-297 by Fc specific endoglycosidase EndoS2 (Genovis) or EndoS (New England Biolabs) which hydrolyze the Fc glycans to the innermost N-acetylglucosamine (GIcNAc) moiety on several subclasses and species of IgG. The enzymes remove all glycoforms, including; high-mannose, hybrid, complex, and bisecting type glycans. 26 μl GlycINATOR resin (Genovis) was transferred to a Mobicol “F” spin column (MoBiTec) mounted in a 1.5 ml Eppendorf tube. The wash of the resin was carried out by attaching a syringe containing 2 ml TBS. The resin was drained by a short centrifugation (1000 g, 20 seconds). The antibody binding was carried out by adding 30 μl of the antibody solution to the resin and incubated for 3 hours at room temperature. Mounting the Mobicol column in a fresh and clean 1.5 ml Eppendorf tube was used to recover the digested antibody by a short centrifugation (1000 g, 20 seconds). To recover the last traces of the antibody, 10 μl of TBS was added to the resin and centrifuged briefly (1000 g, 20 seconds). The pooled antibody solution should have a volume of approximately 40 μl.

An azide-containing Uridine 5′-diphospho-N-acetylazidogalactosamine disodium salt (UPD-GaINAz) was enzymatically attached to the exposed GIcNAc moiety using the β-1,4-Galactosyltransferase Y289L*(GaIT) to generate an azide-activated antibody that is reactive with an alkyne-carrying label. 0.5 μl of the buffer additive (Genovis) was added to the antibody solution. The GaIT (Y2898L) enzyme was dissolved in 20 μl TBS and 1.3 μl of the dissolved GaIT was added to the antibody solution. 3 μl of the reconstituted UDP-GaINAz (in 40 μl TBS) was added to the antibody solution. The solution was mixed by pipetting up and down and incubated overnight protected from light at 30° C.

To remove free UPD-GaINAz, 450 μl of TBS was added to the antibody GaIT reaction and transferred into a 10 kDa centrifugal ultrafiltration unit (Amicon) mounted in a collection tube. The unit was centrifuged at 14,000 g for 5 min at 4° C. and the flow-through discarded. The azide modified antibody sample was washed by adding 500 μl TBS, and centrifuged at 14,000 g for 20 min at 4° C., discarding the flow-through. The sample was recovered by turning the unit upside down in a fresh collection tube and centrifuged briefly (1000 g, 20 sec).

To conjugate the oligonucleotide, 3 μl 1 mM dibenzocyclooctyne conjugated oligonucleotide (5′-DBCO-PEG4 oligonucleotide) was added to the antibody solution (20-30 μl) facilitating the replacement of the cleaved asn-297 glycan moiety with the DNA. As the Kd of the DNA binding to the C′E loop or/and Cγ2-Cγ3 domain is the micromolar range an effective concentration in the submillimolar-millimolar range is necessary for full replacement. The mixture was incubated overnight at 4° C. protected from light. The low bimolecular rate constants of the chemoselective ligation reaction is thought to be overcome as it is preceded by a fast, non-covalent association of oligonucleotide.

To detect the efficiency of the antibody labeling, the free oligonucleotide was removed and the efficiency of the conjugated antibody determined by SDS-PAGE based electropherogram analysis. 100 μl of Pierce™ Protein A/G agarose resin (ThermoFisher Scientific) was loaded into a Mobicol Spin Column. The resin was washed with 2 ml TBS and briefly centrifuged (1000 g, 20 seconds) to drain the resin. The total antibody-oligonucleotide mixture was loaded onto the resin and incubated for 30 minutes at room temperature. After incubation, the resin was washed with 20 ml of PBS and briefly centrifuged. The antibody was eluted by adding 50 μl of elution buffer (pH 3), according to the manufacturer, to the resin and incubating at room temperature for 10 minutes. The tube was briefly centrifuged (1000 g, 20 seconds) and the flow-through collected in the pH neutralising buffer. The labeling efficiency of the antibody with the oligonucleotide is determined using the Agilent Protein 230 Chip on the Bioanalyzer (Agilent) (according to the manufacturer instructions).

As a comparison covalent non-selective conjugation of antibodies was carried out using Thunder-Link® PLUS Oligo Conjugation System (Expedeon—antibody conjugation service provided by the manufacturer). As shown in FIG. 2 this results in approx. 10% of labeling efficacy under manufacturing conditions. Oligonucleotide conjugation of MW Marker—lane 1, pertuzumab unconjugated antibody—lane 2, pertuzumab oligonucleotide conjugate—lane 3, trastuzumab unconjugated antibody—lane 4, trastuzumab oligonucleotide conjugate—lane 5. 4-12% Bis-Tris SDS-PAGE gel, samples reduced and ran in MOPS buffer. Left—SYBR Gold staining detecting oligonucleotide DNA, right—Instant Blue staining detecting protein. The estimated labeling efficacy is approx. 10% (based on SDS-PAGE Agilent Protein 230 Chip run on the Bioanalyzer 2100).

As shown in FIG. 3 the DNA conjugated trastuzumab antibody was analyzed using SDS-PAGE Agilent Protein 230 Chip on the Bioanalyzer. Horizontal scale is kD (kiloDalton). The peak at 26.5 is the antibody light chain, while peak of 53.7 is the antibody heavy chain. The labeled heavy chain has a molecular weight of 69.6 kD, which is in agreement with calculated molecular mass. According to Table 1. of the run of SDS-PAGE Agilent Protein 230 Chip on the Bioanalyzer (from FIG. 3.). The calculated labeling efficacy is 99.3% (1-[peaks_{53.7}/[peaks_{53.7} peak_{69.6}]]=0.993) for a single heavy chain, the labelling efficiency of the whole antibody is 99.995%, which practically considered to be 100%.

TABLE 1
Calculation of the labeling efficacy using
quantitative electrophoretogram data

| Size [kDa] | Rel. Conc. [ng/μl] |
|---|---|
| 4.5 | 0 |
| 7.1 | 0 |
| 9.9 | 0 |
| 18.6 | 22 |
| 26.5 | 1.161.10 |
| 39.4 | 4.1 |
| 53.7 | 197.1 |
| 62.9 | 101.8 |
| 69.6 | 2.585.30 |
| 97.6 | 4.2 |
| 103.4 | 8.3 |
| 240 | 60 |

### Example 2

### Antibody Preparation

100-150 μg of a trastuzumab IgG antibody was used for the process. The antibody was rebuffered into RNAse-free, ultrapure water (UPW) (Fisher Scientific, 2436574), using a 100 kD Ultrafiltration Column (Sigma, UFC510096), a volume of 450 μl of UPW was added and centrifuged at 14,000 g for 10 min at 4° C., the flow-through is discarded. The process was repeated once more. The column is turned upside down into a new collection tube and the antibody was recovered by a short spin (1,000 g, 20 s). The volume of the flow-through is about 20-30 μl.

### Deglycosylation: Modification of the Carbohydrates on the Fc Domain of the Antibody

The rebuffered antibody is transferred to a 1.5 ml reaction tube and 4 μl EndoS enzyme and 4 μl 10×Glycobuffer (New England BioLabs, P0741L) were added to the antibody. RNAse-free, ultrapure water was added up to 44 μl. The reaction was mixed by pipetting up and down.

The antibody was incubated for 1 hour at 37° C. Afterwards, the deglycosylated antibody was rebuffered in 1×TBS (20 mM Tris-HCL; 150 mM NaCl; pH 7.6). For this, the deglycosylated antibody is added to a 100 kD Ultrafiltration Column. To remove the deglycosylation buffer from the antibody solution a rebuffering step was carried out two times, as a volume of 450 μl of 1×TBS was added, the column was placed into a 2 ml collection tube and centrifuged at 14,000 g for 3 min at 4° C., and repeating these steps once more. The flow-throughs were discarded. The column was turned upside down into a new collection tube and the antibody recovered by a short spin (1,000 g, 20 s). The volume of the flow-through was about 20-30 μl.

MST Binding Assay: With or without Blocking Lysine Primary Amine Residues Using Glycosylated and Non-Deglycosylated Trastuzumab Antibodies

The effect of blocking the primary lysine residues was investigated. The experiment investigates whether the non-covalent binding occurs between antibodies and DNA oligonucleotides based on charged interactions between the positively charged lysines residues in the antibody and the negatively charged ssDNA oligonucleotides. The Monolith Protein Labeling Kit RED-NHS 2nd Generation kit was used following the manufacturer protocols including labeling and dye removal. The dye carries a reactive NHS-ester group that reacts with primary amines (lysine residues) to form a covalent bond. Therefore, the lysine primary amine residues are blocked. RED dyes are suited for Monolith and Monolith NT.

Automated instruments with a RED detector (Nano and Pico) and Dianthus NT.23 series. The ligand oligonucleotide (Name:LIGO 5′-TTTTTGGTGACGATCCCGCAAAATCCAATGATGAGCACTTTTTGCAAGCCTCAGCGACC-3′ 59 bp) (SEQ ID No. 1) was used as indicated in the assay setups.

TABLE 2
MST measurement conditions for non-deglycosylated trastuzumab
antibody binding to 59 bp oligonucleotide

| Assay Type | Fluorescent MST binding assay | |
|---|---|---|
| Target (fluorescent molecule) | | |
| Name: | Trastuzumab antibody | |
| Concentration (constant): | 500 | nM |
| Vol. in final reaction mix: | 5 | μl |
| Ligand (non-fluorescent | | |
| molecule) | | |
| Name: | 59-mer single-stranded | |
| | oligonucleotide (LIGO) | |
| Max. concentration: | 1.00 | mM |
| Min. concentration: | 30.5 | nM |
| Vol. in final reaction mix: | 5 | μl |
| Experimental setup | | |
| Assay buffer: | TBS Buffer | |
| Capillary type: | Standard | |
| MST Instrument: | Monolith NT.115 (red-pico) | |
| Laser Power: | 40% | |
| LED Power: | 30% | |
| Temperature: | 25° | C. |

| | |
|---|---|
| Analysis Method: | MST Signal |
| Type of Repeats: | technical |

Results are shown in FIG. 4.

TABLE 3
MST measurement conditions for deglycosylated trastuzumab
antibody binding to 59 bp oligonucleotide

| Assay Type | Fluorescent MST binding assay | |
|---|---|---|
| Target (fluorescent molecule) | | |
| Name: | EndoS deglycosylated | |
| | trastuzumab antibody | |
| Concentration (constant): | 500 | nM |
| Vol. in final reaction mix: | 5 | μl |
| Ligand (non-fluorescent | | |
| molecule) | | |
| Name: | 59-mer single-stranded | |
| | oligonucleotide (LIGO) | |
| Max. concentration: | 1.00 | mM |
| Min. concentration: | 30.5 | nM |
| Vol. in final reaction mix: | 5 | μl |
| Experimental setup | | |
| Assay buffer: | TBS Buffer | |
| Capillary type: | Standard | |
| MST Instrument: | Monolith NT.115 (red-pico) | |
| Laser Power: | 40% | |
| LED Power: | 30% | |
| Temperature: | 25° | C. |

| | |
|---|---|
| Analysis Method: | MST Signal |
| Type of Repeats: | technical |

Results are shown in FIG. 5.

TABLE 4
MST measurement conditions for non-deglycosylated trastuzumab
antibody binding to 59 bp oligonucleotide

| Assay Type | Fluorescent MST binding assay | |
|---|---|---|
| Target (fluorescent | | |
| molecule) | | |
| Name: | Trastuzumab antibody labeled | |
| | with NT-650-NHS-2nd Gen | |
| Concentration (constant): | 10 | nM |
| Vol. in final reaction mix: | 5 | μl |
| Ligand (non-fluorescent | | |
| molecule) | | |
| Name: | 59-mer single-stranded oligonucleotide | |
| Max. concentration: | 2.25 | mM |
| Min. concentration: | 68.7 | nM |
| Vol. in final reaction mix: | 5 | μl |
| Experimental setup | | |
| Assay buffer: | 7.7 mM Tris pH 7.5, | |
| | 150 mM NaCl, 0.005% Tween-20 | |
| Capillary type: | Standard | |
| MST Instrument: | Monolith NT.115 (red-pico) | |
| Laser Power: | 40% | |
| LED Power: | 8% | |
| Temperature: | 25° | C. |

| | |
|---|---|
| Analysis Method: | MST Signal |
| Type of Repeats: | technical |

Results are shown in FIG. 6.

TABLE 5
MST measurement conditions for deglycosylated trastuzumab
antibody binding to 59 bp oligonucleotide

| Assay Type | Fluorescent MST binding assay | |
|---|---|---|
| Target | | |
| (fluorescent molecule) | | |
| Name: | EndoS deglycosylated trastuzumab | |
| | antibody labeled with NT-650-NHS-2nd Gen | |
| Concentration (constant): | 10 | nM |
| Vol. in final reaction mix: | 5 | μl |
| Ligand (non-fluorescent | | |
| molecule) | | |
| Name: | 59-mer single-stranded | |
| | oligonucleotide | |
| Max. concentration: | 50.0 | μM |
| Min. concentration: | 1.53 | nM |
| Vol. in final reaction mix: | 5 | μl |
| Experimental setup | | |
| Assay buffer: | 7.7 mM Tris pH 7.5, | |
| | 150 mM NaCl, 0.005% | |
| Capillary type: | Tween-20 | |
| MST Instrument: | Standard Monolith NT.115 (red-pico) | |
| Laser Power: | 40% | |
| LED Power: | 8% | |
| Temperature: | 25° | C. |

| | |
|---|---|
| Analysis Method: | MST Signal |
| Type of Repeats: | technical |

Results are shown in FIG. 7.

Result Microscale thermophoresis (MST) was used for the biophysical analysis of interactions between biomolecules. MST was used for a temperature-induced fluorescence change of antibody target as a function of the concentration of a non-fluorescent oligonucleotide ligand. MST measurements were carried out to identify binding abilities between ssDNA oligonucleotides used for labeling of antibodies and the antibodies itself. Under autofluorescent (based tryptophan fluorescence) conditions regarding the antibody target, the oligonucleotide concentrations higher than 0.1 mM show characteristic binding curves and such curves were detected for both glycosylated and non-glycosylated trastuzumab antibodies (FIGS. 4, and 5.). While under conditions where the antibodies lysine residues are blocked such binding curves were not detected (FIGS. 6, and 7.). The experimental results confirm the non-covalent binding between antibodies and DNA oligonucleotides is based on charged interactions between the antibodies (assuming positive charged lysines) and negatively charged ssDNA oligonucleotides.

### Example 3—Alternative Highly Efficient Protocol for Antibody Labeling

Rebuffer 100 μg of trastuzumab antibody.

Briefly, using a 100 kD Ultrafiltration Column (Sigma, UFC510096), a volume of 450 μl of UPW, ultrapure water (UPW) (Fisher Scientific, 2436574), was added and centrifuged at 14,000 g for 3 min at 4° C., the flow-through is discarded. The process was repeated once more. The column is turned upside down into a new collection tube and the antibody was recovered by a short spin (1,000 g, 20 s). The volume of the flow-through is about 20-30 μl. The deglycosylation step was carried out by adding to 20 μl rebuffered antibody 3 μl EndoS (New England BioLabs, P0741L), 3 μl 10×Glyco Buffer (New England BioLabs, P0741L) and UPW to a total volume of 30 μl. The mixture was incubated 1 h at 37° C. wrapped in parafilm. For UDP-GaINAz attachment 250 μl UPW was added in one vial of 2.5 mg UDP-GaINAz and reconstituted for 20 min. The following was added to the deglysolated antibody reaction and mixed: 0.5 μl buffer additive (Actome), 3 μl reconstituted GaINAz and UPW up to total volume of 43.5 μl. After mixing, 1.3 μl GaIT (Actome) was added and mixed, and incubated overnight at 30° C. wrapped in parafilm protected from light.

A 100 kD Ultrafiltration Column (Sigma, UFC510096) was used to remove the UDP-GaINAz. A volume of 400 μl of 1×TBS, 20 mM Tris-HCL; 150 mM NaCl; pH 7.6), was added and centrifuged at 14,000 g for 10 min at 4° C., the flow-through was discarded. The process was repeated once more. The column was turned upside down into a new collection tube and the antibody was recovered by a short spin (1,000 g, 20 s). The volume of the flow-through was about 20-30 μl.

Click chemistry was caused out by adding 2 μl of a 1 mM 5′-DBCO-PEG4-Oligonucleotide to the antibody preparation, mixing by vortexing, then spinning shortly and wrapped in parafilm, incubated overnight at 4° C. 4 μl of the labeled antibody solution was analyzed with the Bioanalyzer 2100 (Agilent) Protein 230 Kit according to the manufacturer instructions. Labeling Efficiency was calculated:

$$LE : ( 1 - {( \frac{{h}_{unlabeled}}{{h}_{unlabeled} + {h}_{labeled}} )}^{2} ) * 100$$

FIG. 9 shows the method produces oligonucleotide labeled trastuzumab with 99.2% labeling efficiency. The peak at 26 kD indicates the light chain, peak at 52.1 kD indicates unlabeled heavy chain of the antibody and the labeled heavy chain of the antibody is at 74 kD.

The same protocol was used for antibody labeling and determine efficiency using different concentrations of 5′-DBCO-PEG4 oligonucleotide, instead of 2 μl 1 mM 5′-DBCO-PEG4 oligonucleotide, experiments were performed using 3 μl (0.15 mM), the default 2 μl (0.1 mM) (see at protocol), 1.5 μl (0.07 mM) and 0.3 μl (0.015 mM) to demonstrate the concentration dependence of labeling effectiveness.

### Result

Using 3 μl (0.15 mM), the default 2 μl (0.1 mM) (see above), 1.5 μl (0.07 mM) and 0.3 μl (0.015 mM) of 1 mM 5′-DBCO-PEG4 oligonucleotide, the concentration dependence of labelling effectiveness was demonstrated supporting the measured MST affinities between antibodies and nucleic acids as shown in FIG. 9. This demonstrates that a high concentration oligonucleotide is necessary for a high labelling efficiency resulting in virtually 100% efficiency of labelling.

1. A process for labelling an antibody with an oligonucleotide, said process comprising the step of
(a) Reacting the antibody with an oligonucleotide conjugated to a reactive group, to form a covalent linkage between the oligonucleotide and the antibody;
wherein the concentration of the oligonucleotide conjugate is at least 1 μM.

2. The process of claim 1, the process comprising the steps of
(a) deglycosylating a conserved N-glycosylated amino acid within a CH2 domain of antibody heavy chain to form a deglycosylated antibody;
(b) Reacting the deglycosylated antibody with an oligonucleotide conjugated to a reactive group to form a covalent linkage between the oligonucleotide and the deglycosylated antibody;
wherein the concentration of the oligonucleotide conjugate is at least 1 μM.

3. The process of claim 1 or claim 2 wherein the reactive group is capable of reacting with a primary amine or sulfhydryl group to form a covalent bond.

4. The process of claim 3 wherein the reactive group capable of reacting with a primary amine is selected from isothiocyanates, isocyanates, acyl azides, NHS esters, sulfonyl chlorides, aldehydes, glyoxals, epoxides, oxiranes, carbonates, aryl halides, imido esters, carbodiimides, anhydrides and fluorophenyl esters.

5. The process of claim 3 wherein reactive group capable of reacting with a sulfhydryl group is selected from haloacetyls, maleimides, aziridines, acrylic alloys, arylation agents, vinyl sulfones, pyridyl disulfides, TNB thiols, and disulfide reducing agents.

6. A process for labelling an antibody with an oligonucleotide comprising
(a) deglycosylating a conserved N-glycosylated amino acid within a CH2 domain of antibody heavy chain;
(b) attaching a functional group to the deglycosylated antibody to form a modified antibody;
(c) Reacting the modified antibody with an oligonucleotide conjugated to a reactive group to form a covalent linkage between the oligonucleotide and the modified antibody;
wherein the reactive group reacts with the functional group to form a covalent linkage; and
wherein the concentration of the oligonucleotide conjugate is at least 1 μM.

7. The process of claim 6 wherein the functional group and reactive group are selected from an azido group and a cycloalkynyl group or alkynyl group; a thiol group and an N— malemide group, iodoacetamide, chloroacteamide or disulfide group; a ketone or aldehyde group and a hydrazine, acylhydrazine, aniline or alkoxyamine.

8. The process of claim 6 wherein the cycloalkynyl group is a cyclooctyne group.

9. The process of any one of claims 6 to 8 wherein a modified GlcNAc moiety is formed by attaching a sugar moiety comprising the functional group.

10. The process of claim 9 wherein the sugar moiety is a galactosamine moiety which is enzymatically attached using a β-1,4-Galactosyltransferase.

11. The process of any preceding claim wherein the antibody is an IgG antibody.

12. The process of claim 11 wherein the N-glycosylated amino acid is Asparagine 297.

13. The process of any preceding claim wherein the oligonucleotide contains 30-150 nucleotides.

14. The process of any one of claims 2 to 13 wherein the deglycosylation is carried out using a Fc specific endoglycosidase.

15. An antibody labelled with an oligonucleotide by a process according to any one of claims 1 to 14.

16. A kit for use in a process as claimed in any of claims 1 to 14 for labelling an antibody with an oligonucleotide comprising:
(i) An oligonucleotide conjugated to a reactive group; and optionally
(ii) A compound comprising a functional group capable of forming a modified GlcNAc moiety; and/or
(iii) A Fc specific endoglycosidase; and/or
(iv) a β-1,4-Galactosyltransferase.


SEQUENCE LISTING (1 sequence)

SEQ ID NO: 1 - DNA, 59 bp, synthetic construct
  source 1..59: mol_type = other DNA
tttttggtgacgatcccgcaaaatccaatgatgagcactttttgcaagcctcagcgacc